(19) United States
(12) Patent Application Publication
(10) Pub. No.: US 2025/0195003 A1
(43) Pub. Date: Jun. 19, 2025

(54) INTRAVASCULAR CATHETER TO MEASURE VESSEL DISTENSIBILITY, COMPLIANCE, AND PULSE WAVE VELOCITY

(71) Applicant: EDWARDS LIFESCIENCES CORPORATION, Irvine, CA (US)

(72) Inventors: Jenna Christine Iaizzo, San Clemente, CA (US); Leonardo Paim Nicolau Da Costa, Tampa, FL (US)

(73) Assignee: EDWARDS LIFESCIENCES CORPORATION, Irvine, CA (US)

(21) Appl. No.: 19/070,872

(22) Filed: Mar. 5, 2025

Related U.S. Application Data
(63) Continuation of application No. PCT/US2023/074870, filed on Sep. 22, 2023.
(60) Provisional application No. 63/377,318, filed on Sep. 27, 2022.

(51) Int. Cl. A61B 5/00; A61B 5/02; A61B 5/024; A61B 5/107; A61B 6/12; A61B 6/50; A61B 90/00; A61M 25/10

(52) U.S. Cl. CPC A61B 5/6859; A61B 5/02007; A61B 5/02444; A61B 5/1076; A61B 5/6853; A61B 6/12; A61B 6/504; A61B 2090/3966; A61B 2560/066; A61B 2562/0219; A61M 25/1002; A61M 25/1011; A61M 2205/0266

(57) ABSTRACT

The devices and methods disclosed herein relate to providing an accurate and direct measure of vessel distensibility. These direct measures can also simultaneously record invasive pressures and provide a precise PWV calculation. The disclosed devices include a catheter that is easily inserted under fluoroscopic guidance through the femoral or radial artery. The disclosed devices enable physicians to diagnose aortic stiffness and to provide a direct measure of aortic compliance.

### CROSS-REFERENCE TO RELATED APPLICATION(S)

This application is a continuation of International Patent Application No. PCT/US2023/074870, filed Sep. 22, 2023, which claims the benefit of U.S. Provisional Patent Application No. 63/377,318 filed Sep. 27, 2022, the complete disclosures of which are hereby incorporated by reference in their entireties for all purposes.

### BACKGROUND

### Field

The present disclosure relates to the field of medical devices and procedures.

### Description of the Related Art

Arterial stiffness is an independent predictor of major adverse cardiovascular events (MACE) such as myocardial infarction and stroke. It is also a consequence of the natural aging process. Pulse wave velocity (PWV) measurements are a well-established way to measure the compliance of major blood vessels, such as the arteries. Aortic and cardiac PWV measurements can be used to evaluate the risk of cardiovascular events.

### SUMMARY

Described herein are methods and/or devices to measure aortic distensibility. The disclosed devices and methods can be configured to simultaneously measure changes in the size of a vessel as well as pulse wave velocity. These measurements can be used to determine vessel distensibility and/or vessel compliance.

In some aspects, the present disclosure relates to an intravascular catheter comprising a first sensor zone extending from the distal end of the catheter and a second sensor zone extending from the body of the catheter, the second sensor zone located a predetermined distance from the first sensor zone, wherein the first sensor zone and the second sensor zone are configured to make distensibility measurements of a vessel in which the catheter is inserted by mechanically measuring the diameter of the vessel through systole and diastole and simultaneously to make pulse wave velocity measurements by measuring a relative timing of pulse waves between the first sensor zone and the second sensor zone.

In some implementations, the intravascular catheter includes more than two sensor zones. In some implementations, the intravascular catheter is configured to measure invasive pressure. In some implementations, the intravascular catheter is configured to capture pressure waveforms. In some implementations, the intravascular catheter is configured to measure flow rate. In some implementations, the first sensor zone includes one or more sensors configured to evaluate properties of a vessel wall. In some implementations, the one or more sensors of the first sensor zone are configured to determine a presence of calcium plaques on the vessel wall. In some implementations, the intravascular catheter is configured for use in arteries and veins. In some implementations, sensors of the first sensor zone include fiber optics. In some implementations, the intravascular catheter includes a lumen configured to deliver a fluid, such as medicine or contrast dye, to a targeted area at or near the first sensor zone or the second sensor zone.

In some aspects, the techniques described herein relate to an intravascular catheter including: a catheter having a body and a distal end; a first sensor zone including a first sensor group, the first sensor group including a plurality of sensors, the first sensor zone configured to extend radially from the body of the catheter at the distal end of the catheter; and a second sensor zone including a second sensor group, the second sensor group including a plurality of sensors, the second sensor zone configured to extend radially from the body of the catheter, the second sensor zone located a predetermined distance from the first sensor zone along the body of the catheter, wherein the first sensor zone and the second sensor zone are configured to make distensibility measurements of a vessel in which the catheter is inserted by mechanically measuring a diameter of the vessel through systole and diastole and simultaneously to make pulse wave velocity measurements by measuring a relative timing of pulse waves between the first sensor zone and the second sensor zone.

In some implementations, the techniques described herein relate to an intravascular catheter, wherein the first and second sensor zones each include a plurality of smaller catheters configured to extend radially away from the body of the catheter to be in intimal contact with the vessel.

In some implementations, the techniques described herein relate to an intravascular catheter, wherein the plurality of sensors of the first sensor group is positioned on the plurality of smaller catheters of the first sensor zone and the plurality of sensors of the second sensor group is positioned on the plurality of smaller catheters of the second sensor zone.

In some implementations, the techniques described herein relate to an intravascular catheter, wherein the plurality of smaller catheters includes a shape memory alloy.

In some implementations, the techniques described herein relate to an intravascular catheter, wherein the first and second sensor zones each include a toroidal balloon that extends radially away from the body of the catheter to be in intimal contact with the vessel.

In some implementations, the techniques described herein relate to an intravascular catheter, wherein the plurality of sensors of the first sensor group is positioned on the toroidal balloon of the first sensor zone and the plurality of sensors of the second sensor group is positioned on the toroidal balloon of the second sensor zone.

In some implementations, the techniques described herein relate to an intravascular catheter further including a sheath configured to cover at least a portion of the body of the catheter at the distal end to cover the first sensor zone and the second sensor zone.

In some implementations, the techniques described herein relate to an intravascular catheter, wherein, in a delivery configuration, the first sensor zone and the second sensor zone are each compressed to be adjacent to the body of the catheter, the first sensor zone and the second sensor zone held adjacent to the body of the catheter by the sheath.

In some implementations, the techniques described herein relate to an intravascular catheter, wherein, in a deployed configuration, the sheath is withdrawn to unsheathe the first sensor zone and the second sensor zone to allow the first sensor zone and the second sensor zone to expand radially.

In some implementations, the techniques described herein relate to an intravascular catheter, wherein the first sensor zone and the second sensor zone include shape memory alloys that self-expand upon being unsheathed.

In some implementations, the techniques described herein relate to an intravascular catheter, wherein the first sensor zone and the second sensor zone each include a toroidal balloon that is inflated after being unsheathed.

In some implementations, the techniques described herein relate to an intravascular catheter, wherein the plurality of sensors of the first and second sensor zones includes accelerometers.

In some implementations, the techniques described herein relate to an intravascular catheter, wherein the plurality of sensors of the first and second sensor zones includes radiopaque markers.

In some implementations, the techniques described herein relate to a method for measuring vessel distensibility, the method including: measuring at a plurality of times a size of an inner diameter of a vessel at a first location; determining a change in size of the inner diameter at the first location based on the measured sizes of the inner diameter at the first location; measuring at a plurality of times a size of an inner diameter of the vessel at a second location; determining a change in size of the inner diameter at the second location based on the measured sizes of the inner diameter at the second location; determining an arrival time of a pulse wave at the first location; determining an arrival time of the pulse wave at the second location; determining a pulse wave velocity based on relative arrival times of the pulse wave at the first and second locations; and determining a vessel distensibility based on the changes in size of the inner diameter of the vessel and the determined pulse wave velocity.

In some implementations, the techniques described herein relate to a method further including determining a vessel compliance based on the changes in size of the inner diameter of the vessel and the determined pulse wave velocity.

In some implementations, the techniques described herein relate to a method, wherein measuring the size of the inner diameter at the first location occurs simultaneously with measuring the size of the inner diameter at the second location.

In some implementations, the techniques described herein relate to a method, wherein measuring the size of the inner diameter at the first and second locations occurs simultaneously with determining the pulse wave velocity.

In some implementations, the techniques described herein relate to a method, wherein measuring the size of the inner diameter is accomplished using images of radiopaque markers at the first and second locations.

In some implementations, the techniques described herein relate to a method, wherein measuring the size of the inner diameter is accomplished using sensor data acquired at the first and second locations, the sensor data provided by a plurality of sensors in intimal contact with an inner wall of the vessel.

In some implementations, the techniques described herein relate to a method, wherein the pulse wave velocity is determined using sensor data acquired at the first and second locations, the sensor data provided by a plurality of sensors in intimal contact with an inner wall of the vessel.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1A illustrates an example catheter, such as an intravascular catheter, with two sensor zones for simultaneously measuring a diameter of a vessel and pulse wave velocity (PWV).

FIG. 1B illustrates the catheter of FIG. 1A inserted into a vessel, such as an artery like the aorta or vein.

FIG. 2A illustrates another example catheter with a first sensor zone at or near a distal end of the body of the catheter and a second sensor zone at a distance, D, from the first sensor zone along the body of the catheter.

FIG. 2B illustrates the catheter of FIG. 2A deployed in a vessel, similar to the catheter of FIG. 1B.

FIG. 3A illustrates another example catheter with a single sensor zone rather than two or more sensor zones as with the catheter of FIG. 1A.

FIG. 3B illustrates another example catheter with five sensor zones.

FIG. 4A illustrates another example catheter with a single sensor zone rather than two or more sensor zones as with the catheter of FIG. 2A.

FIG. 4B illustrates another example catheter with four sensor zones.

FIGS. 5A, 5B, and 5C illustrate an example of transitioning a catheter from a delivery configuration to a deployed configuration.

FIGS. 6A, 6B, and 6C illustrate an example of transitioning a catheter from a delivery configuration to a deployed configuration.

FIG. 7 illustrates a block diagram of an example distensibility measurement system.

FIG. 8 illustrates a flow chart of an example method for measuring vessel distensibility.

### DETAILED DESCRIPTION

The headings provided herein are for convenience only and do not necessarily affect the scope or meaning of the claimed inventive subject matter.

### Overview

Arterial stiffness is an independent predictor of major adverse cardiovascular events (MACE) such as myocardial infarction and stroke. It is also a consequence of the natural aging process. However, the degree of arterial stiffness will vary significantly from individuals within the same age range, and is dependent of several factors such as presence of comorbidities (e.g., hypertension, diabetes, chronic kidney disease, etc.), lifestyle habits, exercise, diet, etc.

A preferred method for measuring arterial stiffness is a pulse wave velocity (PWV) measurement, commonly performed from carotid to femoral sites. It is an indirect measurement of arterial stiffness because it measures the time it takes for a pulse wave to travel a certain distance (e.g., from the carotid to the femoral artery). The faster the pulse wave travels, the stiffer the vasculature is considered to be.

Although this is an important clinical parameter for assessing cardiovascular risk, it is seldomly performed and has very low clinical adoption. There are several concerns with its accuracy and no established cutoff for what a normal value should be. In addition, there are multiple local and regional PWV measurements, and none accurately measures exact aortic stiffness or the vessel's distensibility. Furthermore, with no direct measure, the PWV and image-based distensibility measures have potential for user error and misinterpretation.

Accordingly, to address these and other issues, disclosed herein are devices and methods for providing an accurate and direct measure of vessel distensibility. These direct measures can also simultaneously record invasive pressures and provide a precise PWV calculation. The disclosed devices include a catheter that is readily inserted under fluoroscopic guidance through the femoral or radial artery. The disclosed devices enable physicians to diagnose aortic stiffness and to provide a direct measure of aortic compliance.

The disclosed devices include an intravascular catheter with two or more sensor zones, the separation between two of the sensor zones being a known distance. A first sensor zone extends from a distal end of the catheter and a second sensor zone extends from the body of the catheter, the second sensor zone located the known or predetermined distance from the first sensor zone. The first sensor zone and the second sensor zone are configured to make distensibility measurements of a vessel in which the catheter is inserted by mechanically measuring the diameter of the vessel through systole and diastole. The catheter is also configured to measure PWV (simultaneous with measuring the diameter of the vessel) by measuring a relative timing of pulse waves between the first and second sensor zones.

The disclosed methods including making simultaneous diameter measurements and PWV measurements using two sensor zones on a catheter that are spaced a known distance apart. The diameter measurements including measuring a diameter of a vessel through systole and diastole. The PWV measurements include measuring the relative timing of pulse waves between the first and second sensor zones.

The disclosed devices and methods that measure vessel diameter (and changes in the diameter) and PWV are advantageous for a number of reasons. For example, measuring both PWV and vessel diameter, and determining vessel compliance from these measurements, can improve the accuracy of arterial stiffness determinations. This is due at least in part to the PWV measurement being affected by things other than vessel distensibility, such as the existence of calcium deposits on the vessel wall. Furthermore, the devices and methods directly measure PWV which is advantageous relative to common techniques that estimate distensibility based on indirect PWV measurements. In addition, by including two or more sensor zones, as described herein, vessel compliance can be determined simultaneously at different locations along the vessel. This may be advantageous because a vessel is expected to have different compliance at different locations along the vessel, such as in the aorta. If the compliance at the different locations differs from what is expected, the deviation from expectation may be used by a clinician as an indicator of a potential problem.

In some implementations, the catheters disclosed herein can be inserted into arteries or veins. For example, the disclosed catheters can be used in the inferior vena cava (IVC) or superior vena cava (SVC) to calculate vessel diameters and flow alterations in relationship to the cardiac and/or respiratory cycle. In some implementations, the catheters disclosed herein can include one or more sensors configured to capture invasive pressure readings and/or pressure waveforms. In some implementations, the catheters disclosed herein include one or more sensors configured to measure flow in the vessel in which the catheter is positioned. The flow can be measured across specified sensor zones (e.g., sensor zones that include a flow sensor or that are configured to respond to flow). In some implementations, the disclosed catheters include different sensor features at the tips of flowering sensor arms or toroidal balloons to evaluate properties of a vessel wall properties, such as the presence of calcium plaques. This can be done with ultrasound, bioimpedance or additional sensor capabilities due at least in part to the calcium providing a different signal than the one offered by the natural vessel wall. In some implementations, the disclosed catheters are configured to acquire measurements gated to the respiratory cycle to reduce or minimize reading errors. This feature may be beneficial when the disclosed catheters are positioned on the venous side as the venous side may vary significantly with inspiration and expiration. In some implementations, the disclosed catheters include a lumen for contrast injection and/or drug administration. In some implementations, the disclosed catheters are delivered using standard transcatheter techniques, including being advanced over a guidewire when navigating inside the vessels.

### Example Catheters to Measure Vessel Distensibility

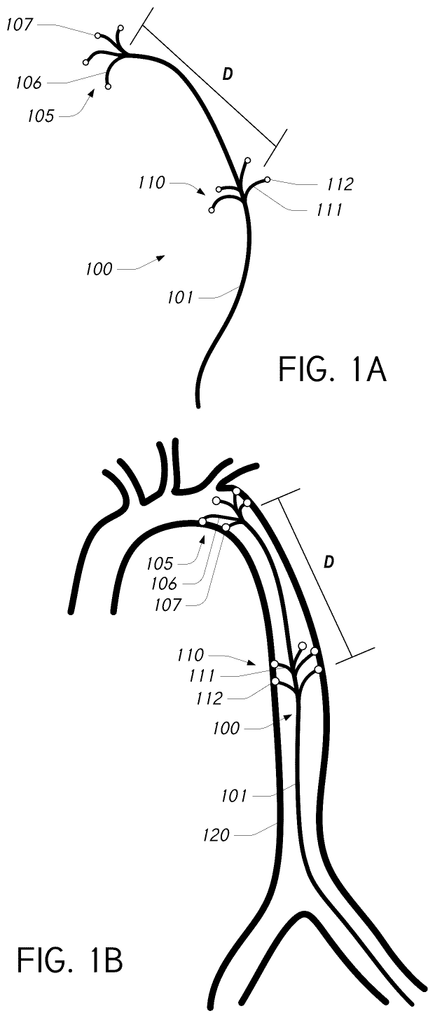

FIG. 1A illustrates an example catheter 100, such as an intravascular catheter, with two sensor zones for simultaneously measuring a diameter of a vessel and PWV. The catheter 100 includes a first sensor zone 105 located at or near a distal end of the catheter 100 and a second sensor zone 110 located a known distance, D, from the first sensor zone 105 along a body 101 of the catheter 100. The catheter 100 includes a plurality of sensors in each of the first sensor zone 105 and the second sensor zone 110.

The first sensor zone 105 is located at or near a distal end of the catheter 100. At the distal end of the catheter 100, the catheter 100 flowers into a plurality of smaller catheters or tubes, referred to as a first plurality of radial arms 106. Individual radial arms of the first plurality of radial arms 106 are equipped with sensors (referred to as a first sensor group 107) that mechanically measure the diameter of a vessel through systole and diastole.

The second sensor zone 110 is located at a specified distance, D, along the body 101 of the catheter 100 from the first sensor zone 105. At the location of the second sensor zone 110 along the body 101 of the catheter 100, the catheter 100 flowers into a plurality of smaller catheters or tubes, referred to as a second plurality of radial arms 111, similar to the first plurality of radial arms 106. Individual radial arms of the second plurality of radial arms 111 are equipped with sensors (referred to as a second sensor group 112) that mechanically measure the diameter of the vessel through systole and diastole, similar to the first plurality of radial arms 106.

The configuration of the first sensor zone 105 and the second sensor zone 110 advantageously enables the catheter 100 to make a direct measurement of the vessel, which may reduce or eliminate potential human error relative to other indirect methods for measuring vessel compliance (e.g., arterial stiffness). Additionally, because the distance between the first sensor zone 105 and the second sensor zone 110 is known, it is possible to directly measure PWV by analyzing the time it takes for a pulse wave to travel from one sensor zone to the other. For example, an algorithm can be implemented that takes the measurements of the first sensor group 107 and the second sensor group 112 and automatically computes the PWV based at least in part on the relative timing of pulses detected or measured by the first and second sensor groups 107, 112.

The first sensor group 107 includes a plurality of sensors with individual sensors coupled to radial arms of the first plurality of radial arms 106. The second sensor group 112 includes a plurality of sensors with individual sensors coupled to radial arms of the second plurality of radial arms 111. In a delivery configuration, the first plurality of radial arms 106 with the first sensor group 107 and the second plurality of radial arms 111 with the second sensor group 112 can be sheathed or covered. In the delivery configuration, the first plurality of radial arms 106 can be adjacent to the body 101 of the catheter 100 or can extend along a longitudinal axis of the body 101 of the catheter 100 being constrained in a group a diameter similar to the diameter of the body 101 while constrained. Similarly, in the delivery configuration, the second plurality of radial arms 111 can be adjacent to the body 101 of the catheter 100. The first plurality of radial arms 106 and the second plurality of radial arms 111 can be constrained by a sheath or cover in the delivery configuration.

In a deployed configuration, the first plurality of radial arms 106 with the first sensor group 107 and the second plurality of radial arms 111 with the second sensor group 112 can be unsheathed or exposed (the deployed configuration being illustrated in FIG. 1A). In the deployed configuration, the first plurality of radial arms 106 and the second plurality of radial arms 111 extend radially from the body 101 of the catheter 100. The first plurality of radial arms 106 and the second plurality of radial arms 111 can curve away from the body 101 to extend radially from the body 101. Sensors of the first sensor group 107 are located at or near distal ends of the first plurality of radial arms 106 and sensors of the second sensor group 112 are located at or near distal ends of the second plurality of radial arms 111.

In other words, in a delivery configuration, the first sensor zone 105 and the second sensor zone 110 are each compressed to be adjacent to the body 101 of the catheter 100, the first sensor zone 105 and the second sensor zone 110 held adjacent to the body 101 of the catheter 100 by a sheath or cover. In a deployed configuration, the sheath or cover is withdrawn to unsheathe the first sensor zone 105 and the second sensor zone 110 to allow the first sensor zone 105 and the second sensor zone 110 to expand radially.

FIG. 1B illustrates the catheter 100 inserted into a vessel 120, such as an artery like the aorta or vein, in a deployed configuration with the first plurality of radial arms 106 extending so that the first sensor group 107 is near or in contact with an inner wall of the vessel 120. Similarly, the second plurality of radial arms 111 extends away from the body 101 of the catheter 100 so that the second sensor group 112 is near or in contact with the inner wall of the vessel 120. Thus, the second sensor group 112 is near or in contact with the inner wall of the vessel 120 a known distance, D, from where the first sensor group 107 is near or in contact with the inner wall of the vessel 120.

In the deployed configuration, the first plurality of radial arms 106 is configured to extend radially from the body 101 to contact the inner wall of the vessel 120. Similarly, in the deployed configuration, the second plurality of radial arms 111 is configured to extend radially from the body 101 to contact the inner wall of the vessel 120. Thus, in the deployed configuration within the vessel 120 the first sensor zone 105 is the known distance, D, from the second sensor zone 110 along the vessel 120.

By way of example, the catheter 100 is configured to be inserted in the aorta 120 so that the first sensor zone 105 and the second sensor zone 110 are each in the aorta 120. Once the catheter 100 is in place within the aorta 120, the first sensor zone 105 and the second sensor zone 110 are unsheathed and the first plurality of radial arms 106 and the second plurality of radial arms 111 flare out to establish different contact points with the inner wall of the aorta 120.

The number of radial arms, or flowering arms, in the first plurality of radial arms 106 can be at least 2, 3, 4, 5, 6, 7, 8, 9, 10, 11, 12, or more. Similarly, the number of radial arms, or flowering arms, in the second plurality of radial arms 111 can be at least 2, 3, 4, 5, 6, 7, 8, 9, 10, 11, 12, or more. The number of radial arms in the first plurality of radial arms 106 can be the same as or different from the number of radial arms in the second plurality of radial arms 111. Similarly, the number of sensors in the first sensor group 107 can be the same as or different from the number of sensors in the second sensor group 112.

The flowering arms of each of the first plurality of radial arms 106 and the second plurality of radial arms 111 can be extendable from the body 101 of the catheter 100. In some implementations, the flowering arms can be made of a self-expanding material or a shape memory alloy, such as Nitinol. In such implementations, the flowering arms can be configured to cause the sensors to come into (or near) intimal contact with the inner wall of the vessel 120.

In some implementations, individual sensors in the first sensor group 107 and/or the second sensor group 112 are configured to detect movement including vibration and/or acceleration. For example, the sensors can include accelerometers or the like. As another example, the sensors can include piezoelectric sensors (e.g., using crystals or ceramic materials).

In some implementations, individual sensors in the first sensor group 107 and/or the second sensor group 112 include radiopaque markers. The radiopaque markers can be tracked using imaging techniques, including fluoroscopy. In such implementations, the absolute and/or relative positions of the sensors in the first sensor group 107 and the second sensor group 112 can be tracked by imaging the sensors and using various imaging techniques to determine the positions of the individual sensors over time.

In some implementations, the first sensor group 107 and the second sensor group 112 can include a mix of movement sensors (e.g., accelerometers) and radiopaque markers. In some implementations, an individual sensor can include both a movement sensor and a radiopaque marker. In some implementations, an individual sensor can include either a movement sensor or a radiopaque marker. In some implementations, the number of sensors in the first sensor group 107 and/or the second sensor group 112 is at least 2, 3, 4, 5, 6, 7, 8, 9, 10, 11, or 12.

In some implementations, and as described herein, measurement systems can be implemented that receive data, signals, measurements, etc. from the sensors of the first sensor group 107 and the second sensor group 112 to calculate a diameter of the vessel 120 during systole and diastole and to calculate PWV in the vessel 120. In some implementations, the disclosed measurement systems can be implemented that receive data, signals, measurements, etc. from imaging devices that record images over time of the sensors of the first sensor group 107 and the second sensor group 112 to calculate a diameter of the vessel 120 during systole and diastole and to calculate PWV in the vessel 120. These measurements and/or calculations can then be used to determine vessel distensibility or compliance (e.g., arterial stiffness). In some implementations, the disclosed measurement systems can be implemented that receive data, signals, measurements, etc. from a combination of the sensors of the first sensor group 107, the sensors of the second sensor group 112, and an imaging device that records images of the sensors of the first sensor group 107 and the second sensor group 112 to calculate a diameter of the vessel 120 during systole and diastole and to calculate PWV in the vessel 120. These measurements and/or calculations can then be used to determine vessel distensibility or compliance (e.g., arterial stiffness). PWV can be determined by measuring a relative timing of pulse waves between the first sensor zone 105 and the second sensor zone 110. In some implementations, one or more pressure sensors are included in the catheter 100. The one or more pressure sensors can be included in the first sensor zone 105, the second sensor zone 110, and/or between the first sensor zone 105 and the second sensor zone 110.

The first sensor group 107 and the second sensor group 112 are configured to make direct distensibility measurements of the vessel 120. For example, the data acquired by the sensors can be used to determine a change in size of the vessel 120 between systole and diastole. This change in size can be used to determine vessel distensibility and/or vessel compliance. In some implementations, the absolute size (e.g., inner diameter) of the vessel 120 can be determined and used to determine vessel distensibility and/or vessel compliance. For PWV measurements, data acquired by the sensors can be used to determine when pulse waves reach the first and second sensor zones 105, 110. Because the distance between the first sensor zone 105 and the second sensor zone 110 is known, the time difference between pulses arriving at the first sensor zone 105 and the second sensor zone 110 can be used to make a direct measurement of PWV. The direct measurement can be made by determining the time it takes for a pulse wave to travel from one sensor zone to the other. Advantageously, this provides a direct measurement of PWC, thereby reducing or eliminating potential human error in the measurement.

In some implementations, the catheter 100 can be configured to measure invasive pressure and/or to capture pressure waveforms. For example, the catheter 100 can include one or more pressure sensors that are part of the first sensor zone 105, the second sensor zone 110, or the body 101.

In some implementations, the catheter 100 can include sensors at the tips of the first plurality of radial arms 106 and/or the second plurality of radial arms 111 that are configured to measure properties of the vessel wall. For example, the sensors can be configured to sense the presence of calcium plaques. This can be accomplished using ultrasound, bioimpedance, or additional sensor capabilities due at least in part to the calcium providing a different signal than the one offered by the natural vessel wall.

In some implementations, the catheter 100 can be configured to measure flow at the first sensor zone 105 and/or the second sensor zone 110. For example, the catheter 100 can include one or more flow sensors that are part of the first sensor zone 105 and/or the second sensor zone 110. In this way, the catheter 100 can be configured to measure flow across the first and second sensor zones 105, 110.

The catheter 100 is configured to be positioned in either veins or arteries. This may be advantageous because the catheter 100 can be used in the IVC or SVC to calculate vessel diameters and flow alterations in relationship to the cardiac and respiratory cycle.

In some implementations, the first sensor group 107 and/or the second sensor group 112 include one or more fiber optics. In some implementations, the catheter 100 includes a lumen through the body 101 for drug administration and/or contrast injection. Thus, the body 101 can include one or more perforations along the body 101, near the first sensor zone 105, and/or near the second sensor zone 110 to allow delivered fluid to exit the catheter 100 at targeted or desired locations.

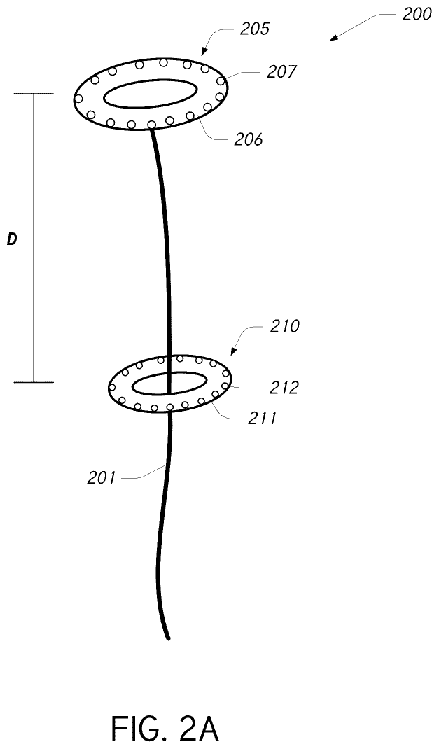

FIG. 2A illustrates another example catheter 200 with a first sensor zone 205 at or near a distal end of the body 201 of the catheter 200 and a second sensor zone 210 at a distance, D, from the first sensor zone 205 along the body 201 of the catheter 200. The first sensor zone 205 comprises a first toroidal balloon 206 that includes a first sensor group 207. The second sensor zone 210 comprises a second toroidal balloon 211 that includes a second sensor group 212. The first sensor group 207 and the second sensor group 212 are similar to the first sensor group 107 and the second sensor group 112, respectively. For example, the sensors can include movement or position sensors, radiopaque markers, or a combination of these.

The catheter 200 is similar to the catheter 100 with a difference being that the catheter 200 substitutes toroidal balloons for flowering radial arms for each of the first sensor zone 205 and the second sensor zone 210. In some implementations, the catheter 200 or the catheter 100 can include one or more sensor zones comprising a toroidal balloon and one or more sensor zones comprising flowering radial arms. In some implementations, the catheter 200 or the catheter 100 can include more than two sensor zones.

FIG. 2B illustrates the catheter 200 deployed in the vessel 120, similar to the catheter 100 deployed in the vessel 120 described herein with reference to FIG. 1B. Once the catheter 200 is in place in the vessel 120, the first toroidal balloon 206 and the second toroidal balloon 211 can be inflated with a liquid, such as saline, to transition the catheter 200 from a delivery configuration to a deployed configuration. In the delivery configuration, the first toroidal balloon 206 and the second toroidal balloon 211 are deflated and can fit within a covering or sheath of the catheter 200. In the deployed configuration, the first toroidal balloon 206 and the second toroidal balloon 211 are configured to come into circumferential contact with an inner wall of the vessel 120. The incompressibility of the liquid within the first toroidal balloon 206 and second toroidal balloon 211 allows pressures and readings to be transmitted to the sensors of the first sensor zone 205 and the second sensor zone 210 with little or minimal error. The central open orifices of the first toroidal balloon 206 and the second toroidal balloon 211 allow blood to pass through the vessel 120 with little or no obstruction of blood flow. The sensors of the first sensor group 207 and the second sensor group 212 are near or are in intimal contact with the inner wall of the vessel 120. In the deployed configuration, the sensors capture direct distensibility measurements of the vessel 120 and PWV, similar to the measurements captured with the catheter 100.

The first toroidal balloon 206 and the second toroidal balloon 211 can be made of a compliant material to reduce or eliminate damage caused to the vessel 120. In some implementations, the catheter 200 includes a feedback mechanism to indicate an inflation level of the first toroidal balloon 206 and the second toroidal balloon 211 and/or to indicate the contact force against the inner wall of the vessel 120 for the first toroidal balloon 206 and the second toroidal balloon 211. This can be done to avoid over-inflating the balloons. This may be desirable to avoid stretching the vessel 120 when inflating the balloons.

The first sensor group 207 can include one or more movement or position sensors and/or one or more radiopaque markers, similar to the first sensor group 107. Likewise, the second sensor group 212 can include one or more movement or position sensors and/or one or more radiopaque markers, similar to the second sensor group 112. The sensors can be attached to the toroidal balloon. The sensors can be embedded in the toroidal balloons. In some implementations, the sensors are configured to extend from the toroidal balloons. In such implementations, the first toroidal balloon 206 and/or the second toroidal balloon 211 are configured to not come into contact with the vessel 120 and the sensors are configured to extend radially out from the toroidal balloons to contact the wall of the vessel 120. In some implementations, the first toroidal balloon 206 and/or the second toroidal balloon 211 are configured to contact the wall of the vessel 120 to anchor the catheter 200 in place to make distensibility and compliance measurements. In some implementations, the number of sensors in the first sensor group 207 and/or the second sensor group 212 is at least 2, 3, 4, 5, 6, 7, 8, 9, 10, 11, or 12. In some implementations, one or more pressure sensors are included in the catheter 200. The one or more pressure sensors can be included in the first sensor zone 205, the second sensor zone 210, and/or between the first sensor zone 205 and the second sensor zone 210.

Similar to the catheter 100, the catheter 200 is advantageous because it combines PWV and distensibility sensors. Algorithms can be implemented that automatically compute PWV based on the timing of sensed pulse waves between the first sensor zone 205 and the second sensor zone 210. Thus, PWV can be directly determined.

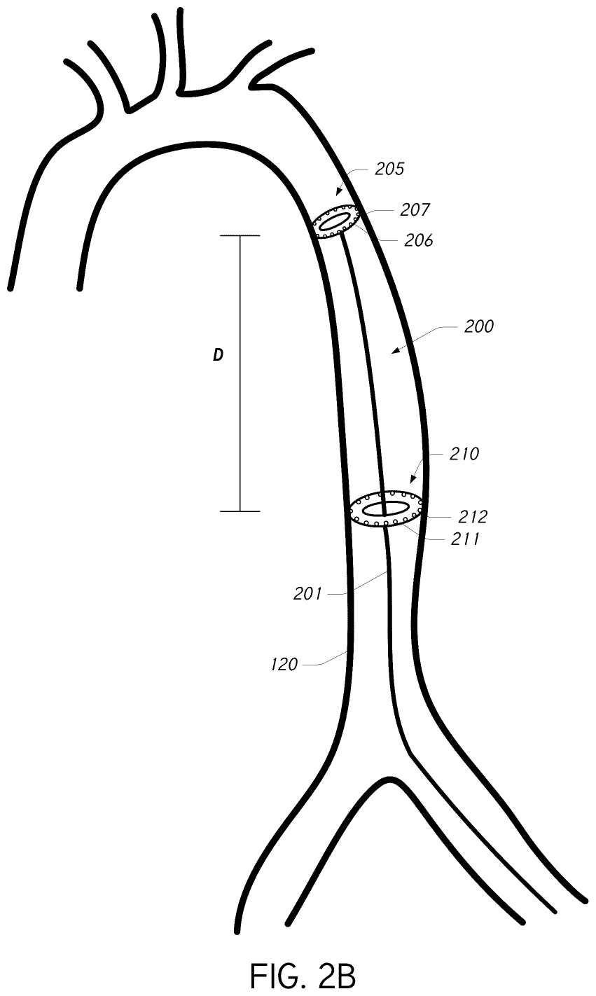

FIG. 3A illustrates another example catheter 300a with a single sensor zone 310 rather than two or more sensor zones as with the catheter 100. In other respects, the catheter 300a is similar to the catheter 100. The sensor zone 310 includes a plurality of radial arms 311 that are each configured to extend radially from a body 301 of the catheter 300a in a deployed configuration, the plurality of radial arms 311 including a sensor group 312 comprising a plurality of sensors. To make distensibility measurements similar to the catheter 100, the catheter 300a can be deployed in a first location where data is acquired. The catheter 300a can then be advanced or withdrawn to move the sensor zone 310 to a second location within the vessel to acquire data at the second location. The data acquired at the two locations can be used to make direct distensibility measurements, e.g., by measuring changes in the inner diameter of the vessel.

FIG. 3B illustrates another example catheter 300b with a five sensor zones 305a-305e. In other respects, the catheter 300b is similar to the catheter 100. Each sensor zone 305a-305e includes a plurality of radial arms that are each configured to extend radially from the body 301 of the catheter 300b in a deployed configuration, the plurality of radial arms including a plurality of sensors to form a sensor group for each sensor zone 305a-305e. The catheter 300b can make distensibility measurements similar to the catheter 100, e.g., by measuring changes in the inner diameter of the vessel. In addition, the inclusion of additional sensor zones can provide a more comprehensive reconstruction of the compliance and distensibility of the vessel. In addition, the additional sensor zones can provide the potential of 3D image reconstruction of the entire vessel by having multiple capturing points. In addition, the different sensor zones provide additional timing information to make accurate PWV measurements. It should be noted that although the catheter 300b is illustrated with five sensor zones, more or fewer sensor zones may be implemented. In addition, each sensor zone 305a-305e is positioned along a body of the catheter 300b a known distance from its neighbors.

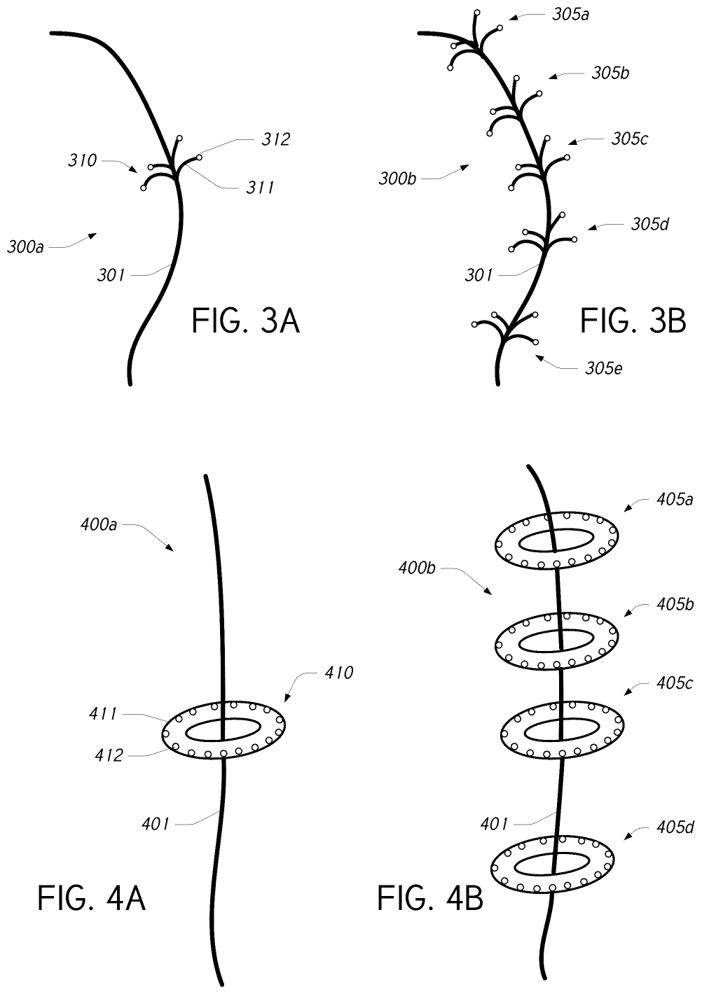

FIG. 4A illustrates another example catheter 400a with a single sensor zone 410 rather than two or more sensor zones as with the catheter 200. In other respects, the catheter 400a is similar to the catheter 200. The sensor zone 410 includes a toroidal balloon 411 that is configured to extend radially from a body 401 of the catheter 400a in a deployed configuration, the toroidal balloon 411 including a sensor group 412 comprising a plurality of sensors. The toroidal balloon 411 is configured to be inflated to transition from a delivery configuration to a deployed configuration, as described herein. To make distensibility measurements similar to the catheter 200, the catheter 400a can be deployed in a first location where data is acquired. The catheter 400a can then be advanced or withdrawn to move the sensor zone 410 to a second location within the vessel to acquire data at the second location. The data acquired at the two locations can be used to make direct distensibility measurements, e.g., by measuring changes in the inner diameter of the vessel.

FIG. 4B illustrates another example catheter 400b with a four sensor zones 405a-405d. In other respects, the catheter 400b is similar to the catheter 200. Each sensor zone 405a-405d includes a toroidal balloon that is configured to extend radially from the body 401 of the catheter 400b in a deployed configuration, the toroidal balloons each including a sensor group comprising a plurality of sensors. The catheter 400b can make distensibility measurements similar to the catheter 200, e.g., by measuring changes in the inner diameter of the vessel. In addition, the inclusion of additional sensor zones can provide a more comprehensive reconstruction of the compliance and distensibility of the vessel. In addition, the additional sensor zones can provide the potential of 3D image reconstruction of the entire vessel by having multiple capturing points. In addition, the different sensor zones provide additional timing information to make accurate PWV measurements. It should be noted that although the catheter 400b is illustrated with four sensor zones, more or fewer sensor zones may be implemented. In addition, each sensor zone 405a-405d is positioned along a body of the catheter 300b a known distance from its neighbors.

Deploying a Catheter with a Plurality of Sensor Zones

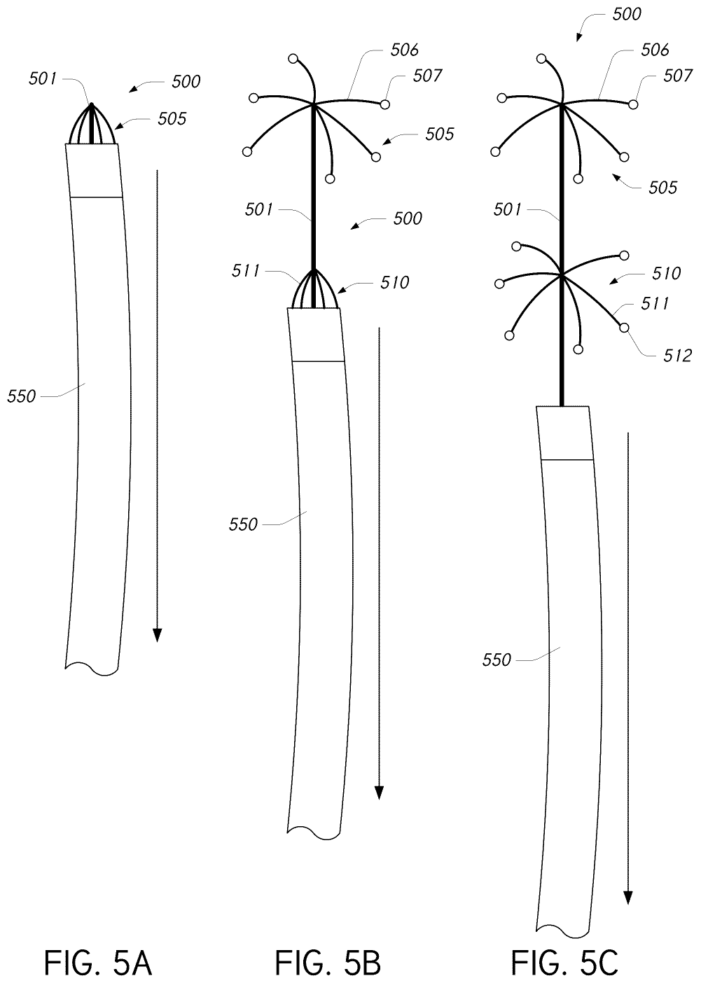

FIGS. 5A, 5B, and 5C illustrate an example of transitioning a catheter 500 from a delivery configuration (e.g., as illustrated in FIG. 5A) to a deployed configuration (e.g., as illustrated in FIG. 5C). The catheter 500 is similar to the catheter 100 described herein with reference to FIGS. 1A and 1B. The catheter 500 includes a first sensor zone 505 comprising a first plurality of radial arms 506 and a first sensor group 507, respectively similar to the first sensor zone 105, the first plurality of radial arms 106, and the first sensor group 107 of the catheter 100. The catheter 500 also includes a second sensor zone 510 comprising a second plurality of radial arms 511 and a second sensor group 512, respectively similar to the second sensor zone 110, the second plurality of radial arms 111, and the second sensor group 112 of the catheter 100.

In a delivery configuration, the first sensor zone 505 and the second sensor zone 510 are covered by a sheath 550 or other covering. In the delivery configuration, the first sensor zone 505 and the second sensor zone 510 are compressed to be adjacent to the body 501 of the catheter 500. In some implementations, the sheath 550 is configured to hold the first sensor zone 505 and the second sensor zone 510 in the compressed or crimped configuration during delivery. In the delivery configuration, the catheter 500 with the sheath 550 is advanced in a transcatheter procedure (e.g., using a transfemoral approach) to a targeted vessel, such as the aorta. Once in a targeted location in the targeted vessel, the catheter 500 can be transitioned to the deployed configuration. To do so, the sheath 550 is withdrawn as shown in FIGS. 5A-5C. Upon unsheathing the first sensor zone 505, as shown in FIGS. 5A and 5B, the first plurality of radial arms 506 flare out to come near or to come into contact with the inner wall of the targeted vessel. The sheath 550 is then further withdrawn to unsheathe the second sensor zone 510, as shown in FIG. 5B. Upon unsheathing the second sensor zone 510, as shown in FIGS. 5B and 5C, the second plurality of radial arms 511 flare out to come near or to come into contact with the inner wall of the targeted vessel a known distance from the first sensor zone 505.

The catheter 500 is configured to be delivered using standard transcatheter techniques. For example, the catheter 500 can be configured to be advanced over a guidewire when navigating inside the vessels.

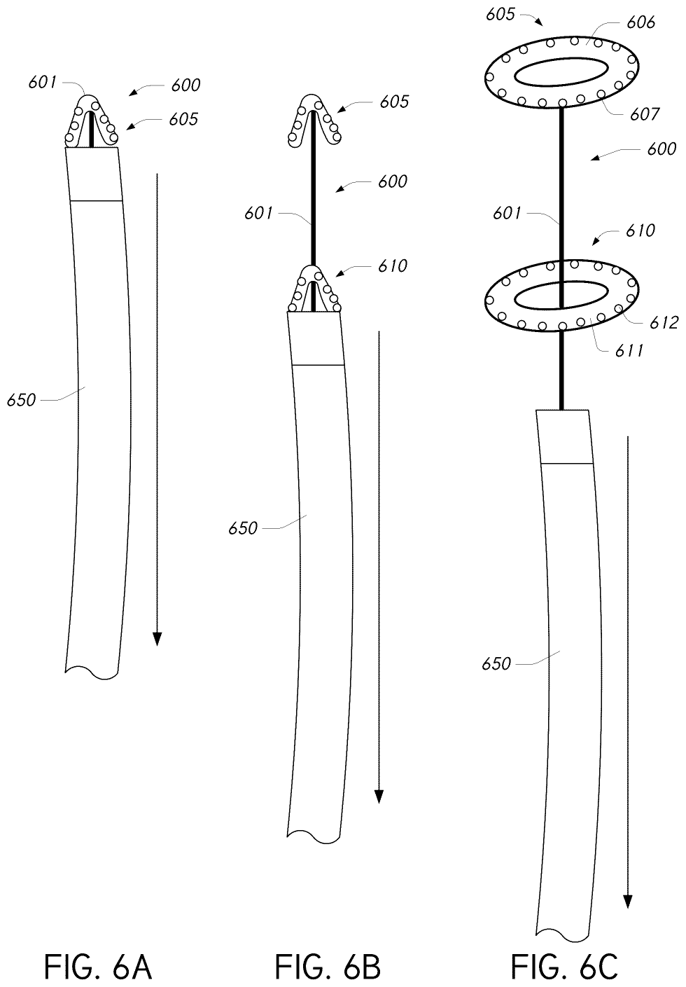

FIGS. 6A, 6B, and 6C illustrate an example of transitioning a catheter 600 from a delivery configuration (e.g., as illustrated in FIG. 6A) to a deployed configuration (e.g., as illustrated in FIG. 6C). The catheter 600 is similar to the catheter 200 described herein with reference to FIGS. 2A and 2B. The catheter 600 includes a first sensor zone 605 comprising a first toroidal balloon 606 and a first sensor group 607, respectively similar to the first sensor zone 205, the first toroidal balloon 206, and the first sensor group 207 of the catheter 200. The catheter 600 also includes a second sensor zone 610 comprising a second toroidal balloon 611 and a second sensor group 612, respectively similar to the second sensor zone 210, the second toroidal balloon 211, and the second sensor group 212 of the catheter 200.

In a delivery configuration, the first sensor zone 605 and the second sensor zone 610 are covered by a sheath 650 or other covering. In the delivery configuration, the first sensor zone 605 and the second sensor zone 610 are compressed to be adjacent to the body 601 of the catheter 600. In some implementations, the sheath 650 is configured to hold the first sensor zone 605 and the second sensor zone 610 in the compressed or crimped configuration during delivery. In the delivery configuration, the catheter 600 with the sheath 650 is advanced in a transcatheter procedure (e.g., using a transfemoral approach) to a targeted vessel, such as the aorta. Once in a targeted location in the targeted vessel, the catheter 600 can be transitioned to the deployed configuration. To do so, the sheath 650 is withdrawn as shown in FIGS. 6A-6C. Upon unsheathing the first sensor zone 605 and the second sensor zone 610, as shown in FIG. 6B, the first toroidal balloon 606 and the second toroidal balloon 611 are inflated using a fluid, such as saline, as shown in FIG. 6C. Inflating the first toroidal balloon 606 causes the first toroidal balloon 606, and the first sensor group 607, to come near or to come into contact with the inner wall of the targeted vessel. Similarly, inflating the second toroidal balloon 611 causes the second toroidal balloon 611, and the second sensor group 612, to come near or to come into contact with the inner wall of the targeted vessel a known distance from the first sensor zone 605.

The catheter 600 is configured to be delivered using standard transcatheter techniques. For example, the catheter 600 can be configured to be advanced over a guidewire when navigating inside the vessels.

### Example Measurement Systems

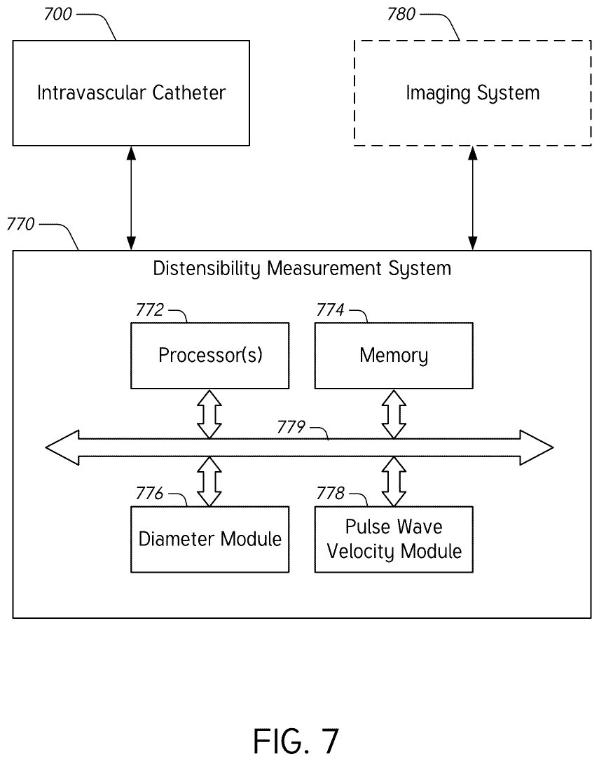

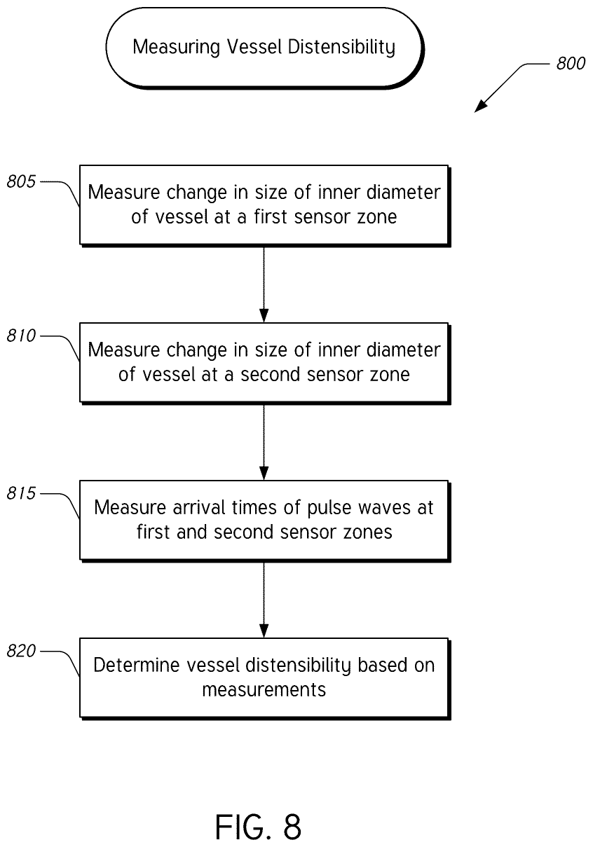

FIG. 7 illustrates a block diagram of an example distensibility measurement system 770. The distensibility measurement system 770 is configured to interface with a catheter 700, such as the catheters 100, 200, 300a, 300b, 400a, 400b, 500, 600 described herein. In some implementations, the distensibility measurement system 770 is also configured to interface with an imaging system 780. The distensibility measurement system 770 is configured to acquire data from the catheter 700 and/or the imaging system 780 and to determine vessel distensibility and/or compliance. In some implementations, the vessel distensibility is determined based at least in part on measurements of the diameter and/or the PWV of the vessel in which the catheter 700 is positioned. The determined vessel distensibility and/or compliance can be used by a clinician during treatment of a patient. The distensibility measurement system 770 can employ any method described herein for determining vessel distensibility, such as the method 800 described herein with reference to FIG. 8.

The distensibility measurement system 770 can include hardware, software, and/or firmware components for measuring distensibility and PWV. The distensibility measurement system 770 includes one or more processors 772, memory 774, a diameter module 776, and a pulse wave module 778. Components of the distensibility measurement system 770 can communicate with one another, with external systems, and with other components of a network using communication bus 779. The distensibility measurement system 770 can be implemented using one or more computing devices. For example, the distensibility measurement system 770 can be implemented using a single computing device, multiple computing devices, a distributed computing environment, or it can be located in a virtual device residing in a public or private computing cloud. In a distributed computing environment, one or more computing devices can be configured to provide the modules 776 and 778 to provide the described functionality.

The distensibility measurement system 770 includes the diameter module 776 to determine and/or track the diameter of the targeted vessel based at least in part on data from the catheter 700. As described herein, the catheter 700 can include one or more sensor zones with sensors positioned at or near an inner wall of the targeted vessel. The data from these sensors can be used by the diameter module 776 to determine changes to the diameter of the targeted vessel through systole and diastole. The changes in diameter can be related to vessel distensibility, which can be determined by the diameter module 776 based at least in part on the determined or measured changes in the size of the targeted vessel. The changes in diameter can be calculated at different points in the targeted vessel corresponding to the different locations of the different sensor zones within the targeted vessel. In some implementations, data from the imaging system 780 is used to determine positions of sensors of a sensor group. This imaging data can be processed to determine sensor locations through systole and diastole to determine changes in vessel size.

The distensibility measurement system 770 includes the pulse wave module 778 to determine and/or track the pulse wave velocity of the targeted vessel based at least in part on data from the catheter 700. As described herein, the catheter 700 can include two or more sensor zones with sensors positioned at or near an inner wall of the targeted vessel. The data from these sensors can be used by the pulse wave module 778 to determine the arrival time of pulses in the targeted vessel. The relative timing of the arrival of pulses can be used to determine PWV in the targeted vessel. The PWV of the targeted vessel is related to the vessel distensibility. Thus, the determined PWV can be used, in conjunction with or independent of the diameter calculations of the diameter module 776, to determine the vessel distensibility and/or compliance. In some implementations, data from the imaging system 780 is used to determine positions of sensors of a sensor group. This imaging data can be processed to determine sensor locations through systole and diastole to determine PWV in the vessel.

The distensibility measurement system 770 is configured to use the data from the sensors of the catheter 700 to detect motion of the sensors of the sensor groups in their respective sensor zones. The motion data can be provided by way of mechanically measuring the motion of the sensors, e.g., using accelerometers. The motion data can be provided by analyzing images of the sensors, e.g., radiopaque markers, to determine the positions of the sensors as a function of time. The catheter 700 can be electrically coupled to the distensibility measurement system 770 using wired or wireless means.

The distensibility measurement system 770 can determine vessel compliance and/or distensibility based on data from one or more measurement sites. In some implementations, the different measurement sites are provided by different sensor zones. In some implementations, the different measurement sites are provided by moving a sensor zone of the catheter 700 to two or more different locations. The distensibility measurement system 770 provides a direct measurement of PWV by measuring pulse arrival times at the two measuring sites. The relative timing of pulses can be determined by measuring the time it takes for a pulse to travel from one measurement site to a neighboring measurement site. The distensibility measurement system 770 provides a direct measure of vessel distensibility by measuring diameter changes in the targeted vessel. The distensibility measurement system 770 can output vessel compliance along with vessel distensibility. In some implementations, the catheter 700 includes one or more pressure sensors. The distensibility measurement system 770 can receive this pressure data from the catheter 700 and can use the pressure data to determine vessel distensibility because there is a relationship between pressure and the changes in size of the vessel through systole and diastole.

The distensibility measurement system 770 includes one or more processors 772 that are configured to control operation of the modules 776, 778 and the memory 774. The one or more processors 772 implement and utilize the software modules, hardware components, and/or firmware elements configured to acquire measurements related to vessel diameter and pulse wave velocity and to determine vessel distensibility and/or vessel compliance. The one or more processors 772 can include any suitable computer processors, application-specific integrated circuits (ASICs), field programmable gate array (FPGAs), or other suitable microprocessors. The one or more processors 772 can include other computing components configured to interface with the various modules and data stores of the distensibility measurement system 770.

The distensibility measurement system 770 includes the memory 774 configured to store configuration data, analysis parameters, control commands, databases, algorithms, executable instructions (e.g., instructions for the one or more processors 772), and the like. The memory 774 can be any suitable data storage device or combination of devices that include, for example and without limitation, random access memory, read-only memory, solid-state disks, hard drives, flash drives, and the like.

In some implementations, the distensibility measurement system 770 is configured to provide feedback to a user. For example, a feedback mechanism can be introduced that receives signals back from the sensors of the catheter 700 indicating contact with the vessel wall. Responsive to a targeted percentage of the sensors indicating contact with the vessel wall, a visual or audible feedback cue can be provided (e.g., a green light) to an operator to indicate satisfactory contact with the vessel wall. The targeted percentage can be configurable by a user or operator or it can be hard-coded into the distensibility measurement system 770.

### Example Methods for Measuring Vessel Distensibility

FIG. 8 illustrates a flow chart of an example method 800 for measuring vessel distensibility. For ease of description, the method 800 is described as being performed by a distensibility measurement system, such as the distensibility measurement system 770 described herein with reference to FIG. 7. However, it should be understood that the method 800 can be performed by any other suitable system or component of a system. Likewise, it should be understood that any portion of the method 800, or any portion of a step in the method 800, can be performed by a different system or component of a system. Moreover, the method 800 can be performed by a single system, a combination of components in a single system, a combination of systems, a combination of components in disparate systems, or the like.

In block 805, the distensibility measurement system measures a change in size of an inner diameter of a vessel at a first sensor zone. The change in size can be determined by measuring the relative or absolute positions of the sensors in the first sensor zone as a function of time. The measured position of the sensors can be determined based on mechanical measurements of the sensors (e.g., using accelerometers). The measured position of the sensors can be determined based on imaging of the sensors (e.g., using radiopaque markers).

In block 810, the distensibility measurement system measures a change in size of an inner diameter of the vessel at a second sensor zone. The change in size can be determined by measuring the relative or absolute positions of the sensors in the second sensor zone as a function of time. The measured position of the sensors can be determined based on mechanical measurements of the sensors (e.g., using accelerometers). The measured position of the sensors can be determined based on imaging of the sensors (e.g., using radiopaque markers). The second sensor zone is located a known distance from the first sensor zone, as described herein.

In block 815, the distensibility measurement system measures arrival times of pulse waves at the first and second sensor zones. The arrival times can be determined by mechanical measurements made by the sensors. The arrival times can be determined using imaging data of the sensors. The pulse can be the pulse caused by the heartbeat of the patient or it can be provided by an external stimulus (e.g., external meaning external to the biological systems of the patient).

In block 820, the distensibility measurement system determines vessel distensibility based on the measurements acquired in blocks 805, 810, and 815. The vessel distensibility can be based on the changes in diameter of the targeted vessel in combination with the PWV of the targeted vessel. In some implementations, the vessel distensibility can be based on the changes in diameter of the targeted vessel. In some implementations, the vessel distensibility can be based on the PWV of the targeted vessel. In some implementations, a first estimate of vessel distensibility can be provided that is based on the changes in diameter and a second estimate of vessel distensibility can be provide that is based on PWV. The method 800 can be used to determine vessel compliance as well as vessel distensibility.

The measurements in blocks 805, 810, and/or 815 can be performed sequentially or simultaneously. Thus, the method 800 can be configured to simultaneously measure both the vessel diameter and PWV. The method 800 can also provide direct PWV measurements and direct distensibility measurements, as described herein. The method 800 can be performed in conjunction with other medical procedures, such as an angiogram. In such instances, the addition of the method 800 to the other medical procedure(s) can have a small impact on the time it takes to perform the procedure. For example, the method 800 may take around 5 minutes to perform using the catheters and distensibility measurement systems disclosed herein.

In some implementations, the measurements are gated by ECG signals. For example, diameter measurements can be made during or at peak systole and during or at peak diastole. In this way, the measurements can be targeted towards the cardiac cycle, measuring the maximum and minimum diameters. It may be advantageous to measure the maximum systolic diameter and compare that to the minimum diastolic diameter because changes in diameter may be particularly valuable. This is because a targeted vessel, such as the aorta, is consistently changing. In addition, it may be advantageous to measure the diameter of the targeted vessel in different places, and to determine the changes in diameter of these places, because the targeted vessel has different diameters in different places along the vessel. In some implementations, the method 800 can be performed to measure different set points along different points in the targeted vessel. In some implementations, the method 800 can be used to provide an accurate measurement of the diameter of the targeted vessel in addition to the changes in diameter of the targeted vessel. Similarly, in some implementations, the measurements are gated by the respiratory cycle to reduce or minimize reading errors. This may be particularly beneficial when the catheter is positioned on the venous side as the venous side can vary considerably with inspiration and expiration.

### Additional Features and Embodiments

Although certain preferred embodiments and examples are disclosed below, inventive subject matter extends beyond the specifically disclosed embodiments to other alternative embodiments and/or uses, and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The above description of embodiments of the disclosure is not intended to be exhaustive or to limit the disclosure to the precise form disclosed above. While specific embodiments, and examples, are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times.

Certain terms of location are used herein with respect to the various disclosed embodiments. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described may be performed in any suitable or desirable temporal order. Furthermore, any of the illustrated and/or described operations or steps may be omitted from any given method or process, and the illustrated/described methods and processes may include additional operations or steps not explicitly illustrated or described.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the subject matter disclosed herein and claimed below should not be limited by the particular embodiments described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that may be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number may also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provides an industry-accepted tolerance for its corresponding term and/or relativity between items. For some industries, an industry-accepted tolerance is less than one percent, while for other industries, the industry-accepted tolerance may be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than approximately +/−1%). Some relativity between items may range from a difference of less than a percentage level to a few percent. Other relativity between items may range from a difference of a few percent to magnitude of differences.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same, related, or unrelated reference numbers. The relevant features, elements, functions, operations, modules, etc. may be the same or similar functions or may be unrelated.

What is claimed is:

1. An intravascular catheter comprising:
a catheter having a body and a distal end;
a first sensor zone comprising a first sensor group, the first sensor group comprising a plurality of sensors, the first sensor zone extending radially from the body of the catheter at the distal end of the catheter; and
a second sensor zone comprising a second sensor group, the second sensor group comprising a plurality of sensors, the second sensor zone extending radially from the body of the catheter, the second sensor zone located a predetermined distance from the first sensor zone along the body of the catheter,
wherein the first sensor zone and the second sensor zone are adapted to make distensibility measurements of a vessel in which the catheter is inserted by mechanically measuring a diameter of the vessel through systole and diastole and simultaneously to make pulse wave velocity measurements by measuring a relative timing of pulse waves between the first sensor zone and the second sensor zone.

2. The intravascular catheter of claim 1, wherein the first sensor zone and the second sensor zone each comprise a plurality of smaller catheters that extend radially away from the body of the catheter to be in intimal contact with the vessel.

3. The intravascular catheter of claim 2, wherein the plurality of sensors of the first sensor group is positioned on the plurality of smaller catheters of the first sensor zone and the plurality of sensors of the second sensor group is positioned on the plurality of smaller catheters of the second sensor zone.

4. The intravascular catheter of claim 2, wherein the plurality of smaller catheters comprises a shape memory alloy.

5. The intravascular catheter of claim 1, wherein the first sensor zone and the second sensor zone each comprise a toroidal balloon that extends radially away from the body of the catheter to be in intimal contact with the vessel.

6. The intravascular catheter of claim 5, wherein the plurality of sensors of the first sensor group is positioned on the toroidal balloon of the first sensor zone and the plurality of sensors of the second sensor group is positioned on the toroidal balloon of the second sensor zone.

7. The intravascular catheter of claim 1 further comprising a sheath that covers at least a portion of the body of the catheter at the distal end to cover the first sensor zone and the second sensor zone.

8. The intravascular catheter of claim 7, wherein, in a delivery configuration, the first sensor zone and the second sensor zone are each compressed to be adjacent to the body of the catheter, the first sensor zone and the second sensor zone held adjacent to the body of the catheter by the sheath.

9. The intravascular catheter of claim 8, wherein, in a deployed configuration, the sheath is withdrawn to unsheathe the first sensor zone and the second sensor zone to allow the first sensor zone and the second sensor zone to expand radially.

10. The intravascular catheter of claim 9, wherein the first sensor zone and the second sensor zone each comprise shape memory alloys that self-expand upon being unsheathed.

11. The intravascular catheter of claim 9, wherein the first sensor zone and the second sensor zone each comprise a toroidal balloon that is inflated after being unsheathed.

12. The intravascular catheter of claim 1, wherein the plurality of sensors of the first sensor zone and the plurality of sensors of the second sensor zone comprise accelerometers.

13. The intravascular catheter of claim 1, wherein the plurality of sensors of the first sensor zone and the plurality of sensors of the second sensor zone comprise radiopaque markers.

14. A system for measuring vessel distensibility and pulse wave velocity, the system comprising:
an intravascular catheter comprising:
a catheter having a body and a distal end; and
a plurality of sensor zones associated with the catheter, each sensor zone comprising a sensor group having a plurality of sensors are radially spaced from the body of the catheter, each sensor zone being spaced from an adjacent zone by a predetermined distance along the body of the catheter; and
a distensibility measurement system adapted to interface with the intravascular catheter, the distensibility measurement system comprising:
memory adapted to store computer-executable instructions; and
one or more processors communicably coupled to the memory and adapted to execute the computer-executable instructions to perform the following:
receive measurements of an inner diameter of a vessel in which the intravascular catheter is inserted, the measurements acquired by the plurality of sensor zones corresponding to a plurality of locations within the vessel;
determine a change in size of the inner diameter of the vessel at the plurality of locations within the vessel based on the measurements of the inner diameter acquired by the plurality of sensor zones determine an arrival time of a pulse wave at each sensor zone of the plurality of sensor zones;
determine a pulse wave velocity based on relative arrival times of the pulse wave at the plurality of sensor zones; and
determine vessel distensibility based on the determined changes in size of the inner diameter of the vessel and the determined pulse wave velocity.

15. The system of claim 14, wherein the distensibility measurement system is further adapted to determine vessel compliance based on the determined changes in size of the inner diameter of the vessel and the determined pulse wave velocity.

16. The system of claim 14 further comprising an imaging system in communication with the distensibility measurement system.

17. The system of claim 16, wherein the imaging system is configured to acquire images of radiopaque markers at each sensor zone of the plurality of sensor zones.

18. The system of claim 17, wherein the distensibility measurement system is further adapted to measure the size of the inner diameter using the acquired images of the radiopaque markers.

19. An intravascular catheter comprising:
a catheter having a body; and
a plurality of sensor zones associated with the catheter, each sensor zone comprising a sensor group having a plurality of sensors are radially extending from the body of the catheter, each sensor zone being spaced from an adjacent zone by a predetermined distance along the body of the catheter,
wherein each sensor zone is adapted to make a plurality of distensibility measurements of a vessel in which the catheter is inserted by mechanically measuring a diameter of the vessel at a plurality of times.

20. The intravascular catheter of claim 19, wherein each sensor zone is adapted to make pulse wave velocity measurements by measuring a relative timing of pulse waves between individual sensor zones of the plurality of sensor zones.
